(19) United States
(12) Patent Application Publication
(10) Pub. No.: US 2020/0397621 A1
(43) Pub. Date: Dec. 24, 2020

(54) MEDICAL TAPE

(71) Applicant: YUGENKAISHA CHOURYU, Shizuoka (JP)

(72) Inventor: Toshifumi HIRASHIMA, Shizuoka (JP)

(21) Appl. No.: 17/006,929

(22) Filed: Aug. 31, 2020

Related U.S. Application Data
(63) Continuation-in-part of application No. 16/097,258, filed on Oct. 28, 2018.

(30) Foreign Application Priority Data
Nov. 1, 2017 (JP) 2017-211857

(51) Int. Cl. A61F 13/02; B29C 39/20

(52) U.S. Cl. CPC A61F 13/0283; A61F 13/024; A61F 13/0246; B29L 2007/007; A61F 13/0269; B29C 39/203; A61F 13/0259

(57) ABSTRACT

In order to provide an elastic medical tape having an especially excellent elastic function and a high effect of alleviating persistent skin irritation during application duration caused by an application-duration shrink force, and applicable to skin that hypersensitively reacts to even insignificant irritation, disclosed is a medical tape for alleviating persistent skin irritation during application duration caused by an application-duration shrink force of an elastic base member of an elastic medical tape, the medical tape including: an elastic base member portion (1) having an elastic function; an adhesive portion (2) having a function of applying and holding the elastic base member portion (1) to skin; and an anti-expansion portion (3) having a function of preventing expansion of the elastic base member portion, the anti-expansion portion (3) overlying the elastic base member portion (1), wherein, after in a manufacturing process, the elastic base member portion (1) and the anti-expansion portion (3) are laminated while the elastic base member portion is in a slacked state or in a not-pulled state, and in the manufacturing process, the elastic base member portion (1) and the anti-expansion portion (3) are laminated while a residual shrink force is not generated in the elastic base member portion (1) in order to reduce the residual shrink force of the elastic base member portion (1), the adhesive portion (2) is applied to skin while preventing expansion of the medical tape, and the anti-expansion portion (3) is then removed.

### TECHNICAL FIELD

The present invention relates to a medical tape for alleviating persistent skin irritation during application duration caused by an application-duration shrink force.

### BACKGROUND ART

In the related art, for medical industries or the like, “a type of elastic bandage (hereinafter, referred to as an “adhesive elastic bandage”) having an elastic base member, an adhesive applied beneath the elastic base member, and an inelastic release sheet placed beneath the adhesive” has been widely employed in order to fix a gauze or a pad to a surface, fix a surgical tool such as an indwelling needle or a catheter, protect a surface of a wound or skin, and the like. The indwelling needle is an injection needle inserted into a vein and fixed to a human body for blood sampling or drip infusion. The indwelling needle is also used in long-term drip infusion or the like around a week.

Such an adhesive elastic bandage has skin followability as a characteristic of the elastic base member in order to alleviate skin irritation caused by a body motion at a brace on the skin during application duration. However, as the skin followability is improved, the bandage easily expands by an insignificant force such as a self-weight in some cases. Therefore, the elastic base member may expand unintentionally during application in many cases. For this reason, a “force of recovering the elastic base member to its initial shape when the elastic base member is used in a pulled state” (hereinafter, referred to as a “recoiling shrink force”) is generated, and this recoiling shrink force acts as a cause of persistent skin irritation during the application duration in many cases. In addition, as a countermeasure for the skin irritation caused by the recoiling shrink force, a manufacturer may inform a warning “Please apply the bandage without pulling because it may cause skin irritation” in the handling manual or the like. However, such a simple warning is not an essential countermeasure for the recoiling shrink force that may be unintentionally generated. If a product may easily expand due to its excellent skin followability, determination on expansion is not easy disadvantageously.

In the adhesive elastic bandage of the prior art, polyurethane fiber mixed with other fibers to provide excellent stretchability and recoverability is selected as a material of the elastic base member. However, recently, a film material such as a polyurethane film having an excellent waterproof property, moisture permeability, and skin followability is selected. Since such a film material has excellent skin followability and the like and has a very thin thickness of approximately 10 μm, it does not independently hold a planar shape and is easily rounded under a natural environment. For this reason, as a support for holding the planar shape, a shape protection cover having stiffness higher than that of the elastic base member of the film material, such as an inelastic plastic film, is provided on the film material in order to hold the planar shape and prevent formation of wrinkles or the like.

In this case, in order to set a film-like elastic base member in an unbent state for preventing slackening, distortion, or the like of the product, the elastic base member is laminated with a support for holding a planar shape in a pulled state. Therefore, a shrink force remains in a part of the elastic base member as much as the pulling force in order to prevent deformation. Hereinafter, the “shrink force remaining in the product during the manufacturing process” will be referred to as a “residual shrink force”. Therefore, in the medical tape having the support for holding the planar shape as described above, that is, the “medical tape having a film-like elastic base member, an adhesive applied beneath the elastic base member, an inelastic release sheet underlying the adhesive, and a support bonded on the elastic base member to hold a planar shape, the support having the residual shrink force in a part of the elastic base member” (hereinafter, referred to as a “support-bonded medical tape”, the residual shrink force exists in the film material included in a part of the elastic base member.

Note that the support-bonded medical tape is applied in the following sequence: 1) remove the release sheet; 2) apply the tape to skin; and 3) remove the support. In addition, the residual shrink force of the support-bonded medical tape is not eliminated even by entirely removing the release sheet. When the tape is applied to skin, and the support is removed, the residual shrink force acts to continuously shrink the skin. Since the skin is shrunken, this force causes persistent skin irritation during the application duration.

Here, the residual shrink force existing in the support-bonded medical tape will be described with reference to FIGS. 1 and 2. FIG. 1 is an explanatory diagram illustrating a state in which a support-bonded medical tape having a bisected support structure is applied to skin, and a support of the thumb side is removed. In order to compare states of the elastic base members underlying the support of the thumb side and the support of the fifth finger side, the support of the thumb side is removed, and the support of the fifth finger side is not removed. While fine wrinkles are observed in the elastic base member of the thumb side applied to skin without the support, no change is observed in the elastic base member of the fifth finger side having the support. FIG. 2 illustrates a state in which the remaining support, that is, the support of the fifth finger side is removed. In FIG. 2, fine wrinkles are also observed in the elastic base member of the fifth finger side. It is conceived that this is because, as the support is removed from the support-bonded medical tape, the elastic base member is released and contracted so as to exhibit fine wrinkles in the elastic base member, and a residual shrink force exists in the elastic base member.

In this manner, in the support-bonded medical tape, the residual shrink force is eliminated by removing the support. Therefore, the residual shrink force existing in the support-bonded medical tape can be observed by applying the medical tape to the skin and removing the support to shrink the elastic base member, so that it is recognized that fine wrinkles appear in the elastic base member. Note that, for skin irritation caused by the residual shrink force, there is no countermeasure proposed to a user. Instead, a handling manual or the like informs a warning “If a symptom such as skin disorder (rash, redness, itching, and the like) appears during use of this product, please stop use and carry out appropriate treatment” in order to prevent a user from understanding an external factor, that is, the residual shrink force existing in the product as a user's constitutive endogenous cause. Therefore, unfortunately, a user is not informed of existence of the skin irritation itself caused by the residual shrink force.

In an adhesive elastic bandage (medical tape) having a very thin elastic base member, some of techniques of the prior art propose medical tapes capable of efficiently alleviating persistent skin irritation during the application duration caused by the residual shrink force, and applicable to skin that hypersensitively reacts to even insignificant irritation (for example, see Patent Document 1). In this technique, persistent skin irritation during the application duration of the support-bonded medical tape caused by the residual shrink force is alleviated. However, in these medical tapes, a synchronous shrink portion is synchronized with shrinkage of the elastic base member portion to reduce the residual shrink force existing in the elastic base member portion by removing a release portion before applying the support-bonded medical tape. Therefore, the elastic base member portion of the support-bonded medical tape easily expands at the time of removing the release portion as the elastic base member of the adhesive elastic bandage does. This may generate a recoiling shrink force disadvantageously. Therefore, in the adhesive elastic bandage and the elastic medical tape including the support-bonded medical tape of the prior art, it is difficult to alleviate the persistent skin irritation during the application duration due to the recoiling shrink force and the residual shrink force caused by applying the elastic medical tape. In addition, there was no countermeasure for addressing such disadvantages. Hereinafter, the recoiling shrink force and the residual shrink force caused by applying the elastic medical tape will be referred to as an “application-duration shrink force”.

### CITATION LIST

[Patent Document]
Patent Document 1: 2017-201564

### SUMMARY OF INVENTION

In view of the aforementioned problems, it is therefore an object of the present invention to provide an elastic medical tape having an especially excellent elasticity and an excellent effect of alleviating persistent skin irritation during the application duration caused by the application-duration shrink force, and applicable to skin that hypersensitively reacts to even insignificant irritation.

In order to address the aforementioned problems, the inventors made diligent efforts over and over and completed the present invention. That is, according the present invention, there is provided a medical tape for alleviating persistent skin irritation during application duration caused by an application-duration shrink force of an elastic base member of an elastic medical tape, the medical tape including: an elastic base member portion having an elastic function; an adhesive portion having a function of applying and holding the elastic base member portion to skin; and an anti-expansion portion having a function of preventing expansion of the elastic base member portion, the anti-expansion portion overlying the elastic base member portion, wherein, in a manufacturing process, after the elastic base member portion and the anti-expansion portion are laminated while the elastic base member portion is in a slacked state or in a not-pulled state, the adhesive portion is applied to skin while preventing expansion of the medical tape, and the anti-expansion portion is then removed.

In the medical tape according to the present invention, since a means for reducing the force of persistently shrinking skin caused by the application-duration shrink force of the elastic base member of the elastic medical tape is functionally incorporated into the medical tape, it is possible to reduce the application-duration shrink force and the force of persistently shrinking skin caused by the application-duration shrink force. In addition, it is possible to alleviate persistent skin irritation during application duration caused by the application-duration shrink force of the medical tape. In addition, it is possible to effectively utilize the elastic function such as skin followability of the medical tape by removing the anti-expansion portion after applying the adhesive portion to skin while preventing expansion of the medical tape. As a result, the medical tape is applicable to skin that hypersensitively reacts to even insignificant irritation. Therefore, it is possible to provide a low-irritation medical tape or patch, or the like. Furthermore, it is possible to prevent inflammation in sebaceous glands or sweat glands, inflammation in the root of hair or its surrounding tissues, a medical instrument pressure-related wound caused by poor blood circulation in skin cells, capillary vessels, or the like due to pressure, and the like. Moreover, since it is possible to avoid a user from erroneously pulling the medical tape, there is no need to inform, in a handling manual, a warning such as “Please apply the tape without expanding (pulling) because it may cause skin irritation” or “If a symptom such as skin disorder (rash, redness, itching, and the like) appears during use of this product, please stop use and carry out appropriate treatment”, which may make a user misunderstand an external factor, that is, the residual shrink force existing in the product as a user's constitutive endogenous cause.

### BRIEF DESCRIPTION OF DRAWINGS

FIG. 1 is an explanatory diagram illustrating a state in which the support of the thumb side is removed in order to check the residual shrink force described in paragraph [0007];

FIG. 2 is an explanatory diagram illustrating a state in which the support of the fifth finger side as a remaining support is removed in the diagram of FIG. 1;

FIG. 3 is a schematic cross-sectional view illustrating a medical tape according to a first embodiment of the invention; and

FIG. 4 is a schematic cross-sectional view illustrating the medical tape according to the first embodiment of the invention, in which an anti-expansion portion 3 and an elastic base member portion 1 are laminated in a slacked state.

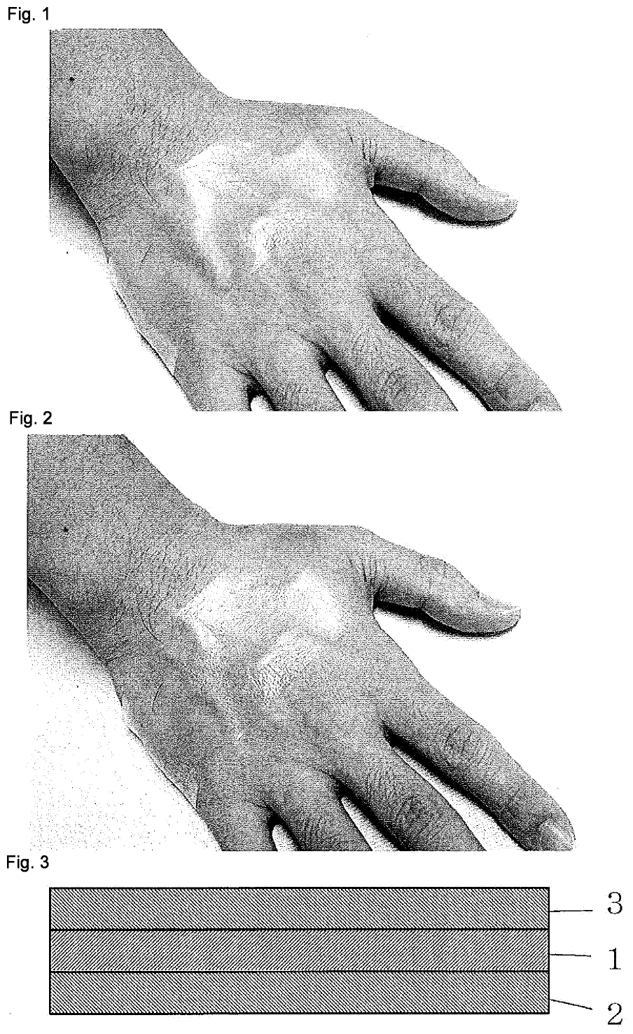

### DESCRIPTION OF EMBODIMENTS

In the course of diligent studies, the inventors found that, although both adhesive elastic bandages and support-bonded medical tapes are elastic medical tapes, they have different structures because presence or absence of the support for maintaining a planar shape is determined depending on whether or not the elastic base member has stiffness for maintaining the planar shape. However, in both structures, a shrink force of the elastic base member that causes persistent skin irritation during application duration, called an application-duration shrink force, acts to continuously shrink skin and generates persistent skin irritation during the application duration.

The skin irritation generated when the elastic medical tape is applied to protect wounds or skin surface will be described. The skin surface has openings of sebaceous glands or sweat glands and also hair. For this reason, the application-duration shrink force of the elastic medical tape continuously deforms openings of sebaceous glands or sweat glands and occasionally acts as an occluding force so as to generate inflammation (such as redness, swelling, fever, pain, and dysfunction) in sebaceous glands or sweat glands in some cases. In addition, the continuous skin shrinking force acts to shrink skin and persistently draw up the hair, so that inflammation occurs in the root of hair or its surrounding tissues in some cases.

Next, skin irritation that may be generated when the elastic medical tape is employed to fix a gauze or pad to a surface or fix a surgical tool such as an indwelling needle or a catheter will be described. The application-duration shrink force of the elastic medical tape not only acts to fix the indwelling needle or the catheter, but also acts to persistently press skin at the portion where the indwelling needle or the catheter is fixed. This force is converted into a force of persistently pressing skin cells, capillary vessels, or the like via such a surgical tool.

Next, the force of persistently pressing skin cells, capillary vessels, or the like will be described. For example, the blood vessels are stretched all over a human body, and its total extension length is approximately 100,000 km, which reaches around two and a half laps of the earth. In addition, nearly 95% of the blood vessels are capillary vessels. The capillary vessels have a diameter of approximately 7 μm and a wall thickness of 1 μm or smaller, which is significantly thin. For this reason, even for an unaware insignificant pressure, the capillary vessels of skin may be easily deformed or obstructed.

There are approximately five million red corpuscles, used to carry oxygen to cells over the entire body, per a microliter of blood. The red corpuscles are disk-shaped solid materials having recessed centers on both sides with a diameter of approximately 7 to 8 μm and a thickness of approximately 2 μm. The red corpuscles pass through the capillary vessels having a diameter of approximately 7 μm while they are deformed. However, since deformation of the red corpuscles as solid materials is limited, it is difficult to allow the red corpuscles to pass through the capillary vessels even when slight deformation occurs in capillary vessels. In addition, the capillary vessels may be clogged due to occlusion of red corpuscles in some cases. As a result, oxygen deficiency often occurs in cells due to poor blood circulation.

Poor blood circulation caused by pressure may generate a disease in which tissues or cells of skin locally die, such as a decubitus, so called a bedsore. Experimentally, it is said that a decubitus is generated if a persistent pressure is applied to the same part of the body for two hours or longer. For this reason, in order to prevent the decubitus in the medical industry, it is recommended that a posture be changed at approximately two hour intervals for a bedridden patient who sleeps in a bed or the like, and at approximately thirty minute intervals for a patient who sits on a wheelchair or the like. In this manner, the decubitus is generated due to poor blood circulation caused by pressure for several hours.

The recent support-bonded medical tapes have been remarkably improved in skin followability, moisture permeability, a waterproof property, skin adherence, and the like, compared to the adhesive elastic bandages of the prior art, so that they can be continuously bonded for about a week. For this reason, the support-bonded medical tapes are used in many cases to fix a gauze or pad to a surface or fix an indwelling needle for drip infusion or the like for about a week. The force of persistently shrinking skin by covering the surgical tool acts to persistently press a portion of the skin where the indwelling needle or the catheter is fixed for several days. Therefore, a medical instrument pressure-related wound may occur in the fixed portion. This is a period of time far exceeding a limitation of several hours for preventing the decubitus.

Referring to a medical staff manual for informing how to apply a medical equipment cord or the like to skin using the tape, there is a warning: “Please be careful! Please do not use a very stretchable tape or elastic bandage as long as possible. Such a tape or bandage will be convenient for covering and fixing the probe, but it is dangerous as it gets tightened gradually after that application. If you are forced to use it, please check the application portion and surrounding condition at shorter intervals.” This shows how dangerous the application-duration shrink force existing in the elastic medical tape is.

As the quality of the adhesive or the elastic base member is improved, the product became durable so as to be fixed for a long time. Furthermore, a product durable even for a week or long has appeared. In such a long-time durable product, it is important to inform a warning “Please gently remove the product along a surface of hair so as not to harm skin” in the handling manual or the like. In order to reduce skin irritation at the time of removing the product, it is effective to provide a means for leaving the tape as it is until it is peeled off without forcibly removing it. However, if the product is left as it is until being peeled off, the application duration naturally becomes long, and continuous skin irritation during the application duration caused by the application-duration shrink force will increase.

Note that, even for insignificant irritation, many patients complain of discomfort and distress. However, if insignificant irritation is persistently applied to the body, “hypaesthesia” which causes dull sensation to a stimulus occurs, so that the number of patients who complain of discomfort or distress is reduced. Although this is generally called “habituation”, it does not mean that the stimulus to the body has disappeared, and deformation or occlusion generated in capillary vessels of skin, poor blood circulation, and the like are improved.

In this manner, in order to reduce persistent skin irritation during the application duration, it is important to reduce a force of persistently shrinking skin caused by a shrink force of the elastic base member of the elastic medical tape. Here, if an anti-expansion portion capable of preventing expansion of the elastic base member portion is provided on the elastic medical tape to prevent pulling of elastic medical tape during application to skin, this is an effective means for alleviating persistent skin irritation during the application duration by reducing the force of persistently shrinking skin due to the recoiling shrink force. In addition, if the elastic base member portion and the anti-expansion portion are laminated without generating a residual shrink force in the elastic base member portion during the manufacturing process of the elastic medical tape, this is an effective means for alleviating persistent skin irritation during the application duration by reducing the force of persistently shrinking skin caused by the residual shrink force of the elastic base member portion.

If such effective means are incorporated into the medical tape, it is possible to alleviate persistent skin irritation during the application duration caused by the application-duration shrink force of the medical tape by reducing the application-duration shrink force and the force of persistently shrinking skin caused by the application-duration shrink force. In addition, it is possible to effectively utilize elastic functions such as skin followability of the medical tape by applying the adhesive portion to skin while preventing expansion of the medical tape and then removing the anti-expansion portion. Therefore, it is possible to apply the medical tape to skin that may hypersensitively react to even insignificant irritation. Moreover, it is possible to prevent a skin trouble, a medical instrument pressure-related wound, or the like caused by persistent skin irritation during the application duration. From this viewpoint, it would be recognized that the elastic medical tapes of the prior art fail to consider the force of persistently shrinking skin caused by the application-duration shrink force of the elastic base member of the elastic medical tape.

In this regard, in order to provide a medical tape having a function of reducing the application-duration shrink force of the elastic base member of the elastic medical tape, the inventors selected an elastic material employable for medical purposes and having excellent performance in the waterproof property, moisture permeability, and skin followability as a material of the elastic base member portion. Then, as a material of the adhesive portion, the inventors selected a medical adhesive causing little skin irritation at the time of application and little adhesive residue or keratin damage at the time of removal. Furthermore, as a material of the anti-expansion portion, the inventors selected a material capable of preventing expansion of the elastic base member portion to produce the medical tape.

The inventors completed the present invention by finding a fact that it is possible to functionally incorporate a means for reducing the force of persistently shrinking skin caused by the application-duration shrink force of the elastic base member of the elastic medical tape into the function of the medical tape by combining the aforementioned materials and laminating on the anti-expansion portion without generating a residual shrink force inside the elastic base member portion, it is possible to alleviate persistent skin irritation during the application duration caused by the application-duration shrink force of the medical tape by reducing the application-duration shrink force and the force of persistently shrinking skin caused by the application-duration shrink force, it is possible to effectively utilize the elastic function such as skin followability of the medical tape by applying the adhesive portion to skin while preventing expansion of the medical tape, and then removing the anti-expansion portion, and it is possible to provide a medical tape applicable to skin that may hypersensitively react to even insignificant irritation.

While examples of the invention will now be described in details with reference to the accompanying drawings, the scope of the invention is not limited thereto. Basically, according to the present invention, materials of the medical tape are appropriately selected and combined, and, in the manufacturing process, the elastic base member portion and the anti-expansion portion are laminated while the elastic base member portion is in a slacked state or in a not-pulled state in order to laminate the elastic base member portion and the anti-expansion portion without generating a residual shrink force in the elastic base member portion. As a result, it is possible to reduce the force of persistently shrinking skin caused by the application-duration shrink force by reducing the application-duration shrink force. In addition, it is possible to alleviate persistent skin irritation during the application duration caused by the application-duration shrink force of the medical tape. Furthermore, it is possible to effectively utilize the elastic function such as skin followability of the medical tape by applying the adhesive portion to skin while preventing expansion of the medical tape, and then removing the anti-expansion portion.

In the manufacturing process according to the present invention, essentially, the elastic base member portion and the anti-expansion portion are laminated without generating a residual shrink force in the elastic base member portion. As a result, it is possible to reduce the force of persistently shrinking skin caused by the application-duration shrink force by reducing the application-duration shrink force. In addition, it is possible to alleviate persistent skin irritation during the application duration caused by the application-duration shrink force of the medical tape. Furthermore, it is possible to effectively utilize the elastic function such as skin followability of the medical tape by applying the adhesive portion to skin while preventing expansion of the medical tape, and then removing the anti-expansion portion.

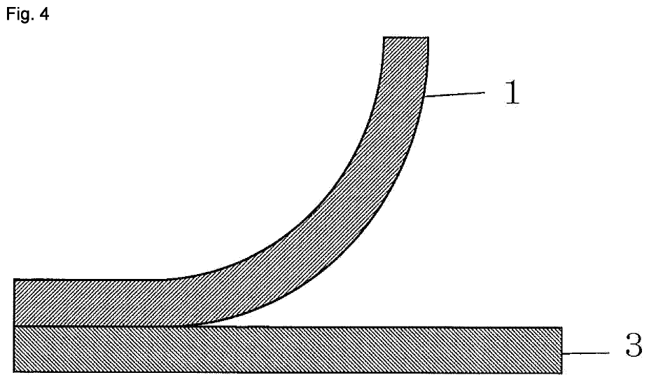

As illustrated in FIGS. 3 and 4, a medical tape according to the first embodiment of the invention includes an elastic base member portion 1 having an elastic function, an adhesive portion 2 having a function of applying and holding the elastic base member portion 1 to skin, and an anti-expansion portion 3 overlying the elastic base member portion 1 to provide a function of preventing expansion of the elastic base member portion 1.

FIG. 3 is a schematic cross-sectional view illustrating a medical tape according to the first embodiment of the invention. Note that reference numeral “1” refers to the elastic base member portion, reference numeral “2” refers to the adhesive portion, and reference numeral “3” refers to the anti-expansion portion. The medical tape according to the first embodiment has the sheet-like anti-expansion portion 3 formed of an inelastic material on the elastic base member portion 1. In addition, the elastic base member portion 1 and the anti-expansion portion 3 are coated with a releasable adhesive. Furthermore, the adhesive portion 2 is provided beneath the elastic base member portion 1. In the manufacturing process, the elastic base member portion 1 and the anti-expansion portion 3 are laminated and fabricated while maintaining a state that does not generate a residual shrink force inside the elastic base member portion.

FIG. 4 is a schematic cross-sectional view illustrating the medical tape according to the first embodiment of the invention, in which the anti-expansion portion 3 and the elastic base member portion 1 are laminated in a slacked state. The anti-expansion portion 3 formed of an inelastic material is stretched, and the elastic base member portion 1 formed of an elastic material is placed on the anti-expansion portion 3 without generating a residual shrink force inside the elastic base member portion. Then, the elastic base member portion 1 is bonded to the anti-expansion portion 3 using a releasable adhesive applied on the anti-expansion portion 3.

In this medical tape, the adhesive portion is applied to skin while preventing expansion of the medical tape. Then, the anti-expansion portion is removed. As a result, it is possible to effectively utilize the elastic function such as skin followability of the medical tape. Therefore, since a means for reducing the force of persistently shrinking skin caused by the application-duration shrink force of the elastic base member of the elastic medical tape is functionally incorporated into the medical tape according to the present invention, it is possible to reduce the force of persistently shrinking skin caused by the application-duration shrink force by reducing the application-duration shrink force. In addition, it is possible to alleviate persistent skin irritation during the application duration caused by the application-duration shrink force of the medical tape. Furthermore, it is possible to effectively utilize the elastic function such as skin followability of the medical tape by applying the adhesive portion to skin while preventing expansion of the medical tape, and then removing the anti-expansion portion. Therefore, it is possible to provide a medical tape applicable to skin that may hypersensitively react to even insignificant irritation.

Specifically, according to the first embodiment of the invention, the elastic base member portion 1 is formed of a cloth used in the adhesive elastic bandage (97% cotton and 3% polyurethane), and a medical adhesive is applied beneath the elastic base member portion 1 to form the adhesive portion 2. In addition, a sheet-like plastic member as the anti-expansion portion 3 having a function of preventing expansion of the elastic base member portion 1 is used as an upper layer of the elastic base member portion 1. Note that, since the elastic base member portion 1 and the anti-expansion portion 3 are laminated without generating the residual shrink force inside the elastic base member portion 1, the elastic base member portion 1 is laminated in a slacked state while maintaining the anti-expansion portion 3 in a stretched state.

According to the first embodiment, the elastic base member portion is formed of a cloth having elastic function (97% cotton and 3% polyurethane). Alternatively, any elastic material may be employed as long as it has a suitable function depending on a use purpose such as for fixing a gauze or pad to a surface, for fixing the surgical tool such as the indwelling needle or the catheter, or for protecting a surface of wounds or skin. For example, a stretchable member such as rubber may be employed. Any other new material may be employed as long as it is suitable for medical purposes. In addition, there is no limitation in the material or the shape. Furthermore, although the adhesive portion is formed of an acrylic adhesive material, any adhesive material may be employed as long as it can be used to apply and hold the elastic base member portion to skin for medical purposes. The adhesive may be applied partially or entirely depending on a use purpose. In addition, there is no limitation in a material or shape of the adhesive. Although the anti-expansion portion is formed of a sheet-like plastic having an inelastic function, any material may also be employed depending on a use purpose without limiting to its material or shape as long as it can prevent expansion of the elastic base member portion.

Although the elastic base member portion and the anti-expansion portion are provided by applying a releasable adhesive, a pseudo adhesive may also be employed. Any other means may also be employed as long as it can be released. In addition, in order to laminate the elastic base member portion 1 and the anti-expansion portion 3 without generating a residual shrink force inside the elastic base member portion 1, the elastic base member portion 1 is laminated in a slacked state while the anti-expansion portion 3 is maintained in a stretched state. Alternatively, a film material such as a polyurethane film may be employed as a material of the elastic base member portion. In addition, the film may be unfolded on a horizontal work table without pulling, and a sheet-like plastic having an inelastic function as a material for the anti-expansion portion or the like may be laminated. Resultantly, any method may be employed as long as the elastic base member portion and the anti-expansion portion are laminated without generating a residual shrink force in the elastic base member portion. In addition, there is no limitation in the laminating means.

Both the medical tapes of FIGS. 3 and 4 according to the present invention have a basic structure of the present invention. In this configuration, a means for reducing the force of persistently shrinking skin cause by the application-duration shrink force of the elastic base member of the elastic medical tape is functionally incorporated into the medical tape. Therefore, it is possible to reduce the force of persistently shrinking skin caused by the application-duration shrink force by reducing the application-duration shrink force. In addition, it is possible to alleviate persistent skin irritation during the application duration caused by the application-duration shrink force of the medical tape. Furthermore, it is possible to effectively utilize the elastic function such as skin followability of the medical tape by applying the adhesive portion to skin while preventing expansion of the medical tape, and then removing the anti-expansion portion. Therefore, it is possible to provide a medical tape applicable to skin that hypersensitively reacts to even insignificant irritation. Moreover, it is possible to prevent a skin trouble caused by persistent skin irritation during the application duration.

While it is assumed that the medical tape has a rectangular shape in the first embodiment described above by way of example, any shape may be freely selected for the medical tape depending on a use purpose. For example, a roll shape or a sheet shape may be employed. Alternatively, a shape matching a respective part of the body may be employed. In addition, a plurality of pieces of the medical tape may be combined in order to match the shape of the respective part. Furthermore, depending on a use purpose or design, a portion having none of the functions of each part of the medical tape may be mixedly provided in a part of the medical tape. A gauze, a pad, or the like may be provided. A release sheet may also be provided or not. Note that, although the medical tape is tightly applied to skin according to the present invention, the medical tape may also be applied by inserting a protection material for protecting skin from the medical adhesive or the like in some cases.

The recoiling shrink force is generated as the elastic medical tape is pulled at the time of application. For this reason, the function of the anti-expansion portion provided to prevent the recoiling shrink force of the medical tape is essentially to reduce the force of persistently shrinking skin caused by the recoiling shrink force. Therefore, the anti-expansion portion is preferably provided on the entire elastic base member portion and preferably acts to prevent expansion of the elastic base member portion in all directions. However, when the medical equipment cord or the like is fixed in an n (omega) connection manner, it is effective to provide a dedicated tape having no anti-expansion portion in a part of the medical tape adjoining a tool or the like in consideration of operability, a fixing force, and the like

Note that the function of the medical tape according to the present invention is effectively applied to a medical tape applied to a human body. For example, since a skin irritation hypersensitivity patient severely reacts to even insignificant skin irritation, the use of medical tapes containing percutaneously absorbable drugs is postponed in some cases. However, since it is possible to alleviate insignificant skin irritation using the medical tape according to the present invention, the medical tape according to the present invention can be used as a patch by blending percutaneously absorbable drugs in the adhesive part.

The medical tape according to the present invention has an extremely high effect of alleviating persistent skin irritation during the application duration caused by the application-duration shrink force so as to be applicable to skin that hypersensitively reacts to even insignificant irritation. In addition, it is possible to prevent a skin trouble or a medical instrument pressure-related wound caused by persistent skin irritation during the application duration. Furthermore, it is possible to provide a low-irritation medical tape, patch or the like. Therefore, the medical tape according to the present invention will greatly contribute to the medical industries.

According to an example of the invention the medical tape includes an elastic base member portion which is a film made of liquid polymer. The medical tape further includes an anti-expansion portion applied on the film after the liquid polymer has been solidified such that after the anti expansion portion is applied the elastic base member is maintained in its slacked state without residual shrink force. That is to say, the method of manufacturing of the medical tape includes forming a film by polymer solution casting. According to the method the polymer is dissolved or dispersed in solution, coated onto a carrier substrate, and then the water or solvent is removed by drying to create a solid layer on the carrier. The resulting cast layer is stripped from the carrier substrate as a film. The film is then used as a base member for the medical tape.

According to the present invention the anti-expansion portion is applied to the film after the film has dried and solidified. This way, the film, which is made as an elastic film, is coupled to the anti-expansion portion when in the slacked state thereof. In other words, instead of laminating the film with the anti-expansion portion during the polymer solution casting process, the lamination is done only after the film is formed. It is noted that when the liquid polymer solidifies to form a solid film, the polymer slightly shrinks. Thus, according to known methods, when the anti-expansion portion is applied while the liquid polymer is still on the carrier substrate, once solidifies shrink forces are formed across the film and are maintained by the anti-expansion portion. Accordingly, the method of the present invention, in which the film solidifies before the anti-expansion portion is applied, allows the polymer to freely shrink such that the film is free on any shrinking forces.

It is pointed out that according to known methods, the anti-expansion portion is applied to the film during the casting process, so as to facilitate the lamination of the base member and the anti-expansion portion. The present invention, however requires that the anti-expansion portion is applied over the already finished film only after the latter is completely dry. Thus, although the manufacturing process of the tape according to the present invention is longer and requires additional steps, the resulting tape provides a base member which is free of any shrining forces.

According to the present invention, since the anti-expansion portion is applied on the film when the latter is completely in its slacked state, and free of any shrining forces, the anti-expansion portion only prevents the film from expanding. Thus, when the medical tape is applied over a skin portion, the film engages the skin while in slacked state, and then the anti-expansion portion is removed. This results in a medical tape which is not expended and does not exert any shrinking forces over the skin.

As described hereinabove the medical tape further includes an adhesive portion having a function of applying and holding the elastic base member portion to skin. The elastic base member portion, which according to the present example is a polymer film, is formed with a first face on which the anti-expansion portion is applied and a second face which is configured to be applied over the skin. In order to facilitate maintaining the medial tape over the skin, the second face can include an adhesive portion.

According to an example, the film is made out of liquid polyurethane, and can further include second hereinabove the medical tape further includes bonded-fiber fabric. The film can include thickness 30 μm or less, so as to avoid stuffiness thereof and to avoid humidity accumulation thereon when applied over the skin. More specifically, according to an example the thickness of the film is between 7 μm to 18 μm, or between 8 μm to 12 μm. Similarly, the anti-expansion portion can have a thickness configured to facilitate peeling thereof after the medical tape is applied over the skin. Thus, the thickness of the anti-expansion portion can be between 5 μm to 15 μm or more specifically between 8 μm to 12 μm.

### REFERENCE SIGNS LIST

1 elastic base member portion
2 adhesive portion
3 anti-expansion portion

1. A method for manufacturing a medical tape, the method comprising:
forming a polymer film by polymer solution casting, said polymer film being configured as an elastic base member portion having an elastic function;
coupling an anti-expansion portion over a first face of said film, said anti-expansion portion being configured for preventing expansion of the elastic base member portion, said step of coupling said anti-expansion portion is carried out after the polymer film is solidified;
applying an adhesive portion over a second face of said film said adhesive portion being configured for holding the film over a skin; and
wherein said step of coupling said anti-expansion portion is carried out while said film is in its slacked state such that said film is free of shrining forces.

2. The method of claim 1 wherein said film has thickness of 30 μm or less.

3. The method of claim 2 wherein said thickness is between 7 μm and 18 μm.

4. The method of claim 3 wherein said thickness is between 8 μm and 12 μm.

5. The method of claim 1 wherein the anti-expansion portion has thickness of between 5 μm and 15 μm.

6. The method of claim 5 wherein said thickness is between 8 μm and 12 μm.

7. The method of claim 1 wherein said polymer film includes polyurethane.

8. The method of claim 1 wherein said polymer film includes bonded-fiber fabric.
